(19) United States
(12) Patent Application Publication
(10) Pub. No.: US 2024/0138819 A1
(43) Pub. Date: May 2, 2024

(54) STOOL SAMPLE COLLECTION AND PROCESSING SYSTEM

(71) Applicant: Tend Health, Inc., Seattle, WA (US)

(72) Inventor: Thomas R. Swetish, Seattle, WA (US)

(21) Appl. No.: 18/547,300

(22) PCT Filed: Feb. 18, 2022
(86) PCT No.: PCT/US22/16947

§ 371 Date: Aug. 21, 2023

Related U.S. Application Data
(60) Provisional application No. 63/151,949, filed on Feb. 22, 2021.

(51) Int. Cl. A61B 10/00

(52) U.S. Cl. CPC A61B 10/0038; A61B 10/0096

(57) ABSTRACT

Disclosed is an apparatus for collection and processing of a sample. The apparatus includes container that can contain a sample, and a capsule carrier assembly that supports a capsule portion. The apparatus further includes a fill nozzle mechanically coupled to the capsule carrier assembly and the container, wherein the fill nozzle provides a passageway for a portion of the sample to be transferred from the container into the capsule portion. A force exertion element exerts a force onto the sample in the container to move the portion of the sample into the capsule portion via the at least one nozzle. The portion of the sample passes through the filter prior to being moved into the capsule portion.

### CROSS-REFERENCE TO RELATED APPLICATIONS

This application claims priority to U.S. Patent Application No. 63/151,949, filed Feb. 22, 2021, entitled “STOOL SAMPLE COLLECTION AND PROCESSING SYSTEM”, the content of which is hereby incorporated by reference herein in its entirety.

### BACKGROUND

Disclosed is a device that allows for the collection and processing of a stool sample. The device is configured for use in mixing, filtration and encapsulation of the sample such as for later oral ingestion or administration such as by rectal or stomal suppository. The device can further be used to collect the entire mixed, filtered, homogenated sample for other uses. It should be appreciated that the device is configured for processing stool samples for a variety of uses and that the examples described herein are not limiting.

A healthy intestinal microbiome has a normal balance of bacteria. A growing body of research supports the importance of an individual's healthy gut microbiome. Various aspects of a person's life can damage the normal balance, which can adversely affect the immune system and how food is processed in the body. Furthermore, disrupted intestinal bacteria balance has been linked to mental health disorders, obesity, malnutrition, and inflammatory diseases.

Persons do not currently have an accessible, reliable way to re-establish a healthy microbiome when it becomes imbalanced (dysbiosis) and many patients are using probiotics that may not yield any benefit or may delay the return a balance of healthy gut bacteria. After disruption of the microbiome, it may be desirable to ingest one's own fecal matter to re-establish a healthy biome or to receive a fecal microbiota transplant from a donor. For fecal matter to be put into a usable form from original raw form, the fecal matter conventionally must be collected and processed through a laborious and time intensive process involving transfer of stool from collection container to a blender, introduction of saline to allow for liquifaction, filtering through strainers or cheese cloth, sometimes centrifugation, followed by reconstitution of the bacterial pellet with a solution of some sort and then transfer into a syringe for use in colonoscopy, into an enema bottle, or into orally ingestible capsules. These steps prevent the ability for a potential point-of-care solution. This conventional process takes multiple hours and typically requires a freezing cycle for later use because these steps prohibit patient administration on the day of this processing burden. Current fecal transplant solutions also rely on a few donors making it impossible or difficult to scale the fecal matter.

### SUMMARY

There is a need for improved devices, kits, and methods for producing fecal transplants, such as in a point-of-care setting. Disclosed is a system for producing capsules that contain a sample, such as a sample of viscous material such as a stool sample. In one aspect, the systems, devices and methods disclosed herein are configured to prepare capsules and/or other forms of a fecal transplant that includes at least a portion of a fecal sample. The capsules may be consumed by a human or animal such as for a fecal transplant.

In one aspect, there is disclosed an apparatus for collection and processing of a stool sample comprising: a container defining a cavity that can contain a stool sample; a capsule carrier assembly defining at least one seat that supports a capsule portion; at least one fill nozzle mechanically coupled to the capsule carrier assembly and the container, wherein the fill nozzle provides a passageway for a portion of the stool sample to be transferred from the container into the capsule portion; and a force exertion element mechanically coupled to the container such that the force exertion element exerts a force onto the stool sample in the container to move the portion of the stool sample into the capsule portion via the at least one nozzle; and a filter positioned relative to the force exertion element, wherein the portion of the stool sample passes through the filter prior to being moved into the capsule portion.

In another aspect, there is disclosed an apparatus for collection and processing of a sample comprising: a container defining a cavity that can contain a sample; a capsule carrier assembly defining at least one seat that supports a capsule portion; at least one fill nozzle mechanically coupled to the capsule carrier assembly and the container, wherein the fill nozzle provides a passageway for a portion of the sample to be transferred from the container into the capsule portion; a force exertion element mechanically coupled to the container such that the force exertion element exerts a force onto the sample in the container to move the portion of the sample into the capsule portion via the at least one nozzle; and a filter positioned relative to the force exertion element, wherein the portion of the sample passes through the filter prior to being moved into the capsule portion.

In another aspect, there is disclosed an apparatus for collection and processing of a stool sample comprising: a container defining a space that can contain a stool sample; at least one capsule carrier assembly configured to support at least one capsule; a cartridge including a piston, a filter, a mixing and dispensing mechanism, and one or more fill nozzles configured to align with an opening of the at least one capsule, wherein the cartridge couples to the at one container; and an actuator that can be actuated to rotate the cartridge.

In another aspect, there is disclosed a method of processing a stool sample, comprising: exerting a force onto a stool sample such that the stool sample passes through a filter of a collection device; causing the stool sample to pass through a plurality of injection nozzles into a plurality of first capsule portions positioned in or on the collection device, such that each first capsule portion of the plurality of first capsule portions contains a portion of the stool sample; attaching a second capsule portion to each of the first capsule portions to form plurality of capsules that each contains a portion of the stool sample.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1A shows a perspective view of a sample collection system and device that is configured to collect, store, mix, filtrate, and/or encapsulate a stool sample.

FIG. 1B shows an exploded view of the system and device of FIG. 1A.

FIG. 1C shows an exploded view of another embodiment of a sample collection device.

FIG. 2A shows an exploded view of an embodiment of capsule carrier assembly of the device.

FIGS. 2B and 2C show components of the capsule carrier assembly.

FIG. 2D shows a sectional view of the capsule carrier assembly.

FIG. 2E shows the capsule carrier of FIG. 2A coupled to the sample collection device.

FIG. 2F shows an exploded view of another embodiment of capsule carrier assembly of the device.

FIG. 2G shows partially transparent view of the embodiment of the capsule carrier assembly of the device.

FIG. 2H shows the capsule carrier assembly of FIG. 2F in a position ready to be coupled to a cartridge of the device.

FIG. 3 shows sample devices pursuant to a stool collection process.

FIG. 4 shows a container of the device.

FIG. 5 shows the container coupled to an embodiment of a cartridge of the device.

FIG. 6 shows a cross-sectional view of the container coupled to the cartridge in an embodiment.

FIG. 7 shows a top view of the cartridge in an alternate embodiment.

FIG. 8 shows the device in an assembled state with a crank in position for actuation in an embodiment.

FIGS. 9 and 10 show enlarged views of portions of the device in an embodiment.

FIGS. 11-14 show various views of an embodiment of the capsule carrier assembly.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only and is not intended to be limiting. Unless defined otherwise, all technical terms used herein have the same meaning as commonly understood by one skilled in the art to which this subject matter belongs.

In an aspect, the present disclosure provides a system for producing fecal transplant capsules. The systems, devices and methods disclosed herein are configured to prepare capsules and/or other forms of a fecal transplant that includes at least a portion of a fecal sample. The capsules may be consumed by a human or animal such as for a fecal transplant. In a non-limiting example, the patient consumes one or more capsules following a course of antibiotics, chemotherapy, or other medical procedure that depletes or decreases gut microbiota. It should be appreciated that the devices described herein can also be used to process and prepare samples of other materials such that the devices are not limited for use with a stool sample. For example, the device can be used to process, mix, filter and/or transfer paste-like or viscous material into capsules.

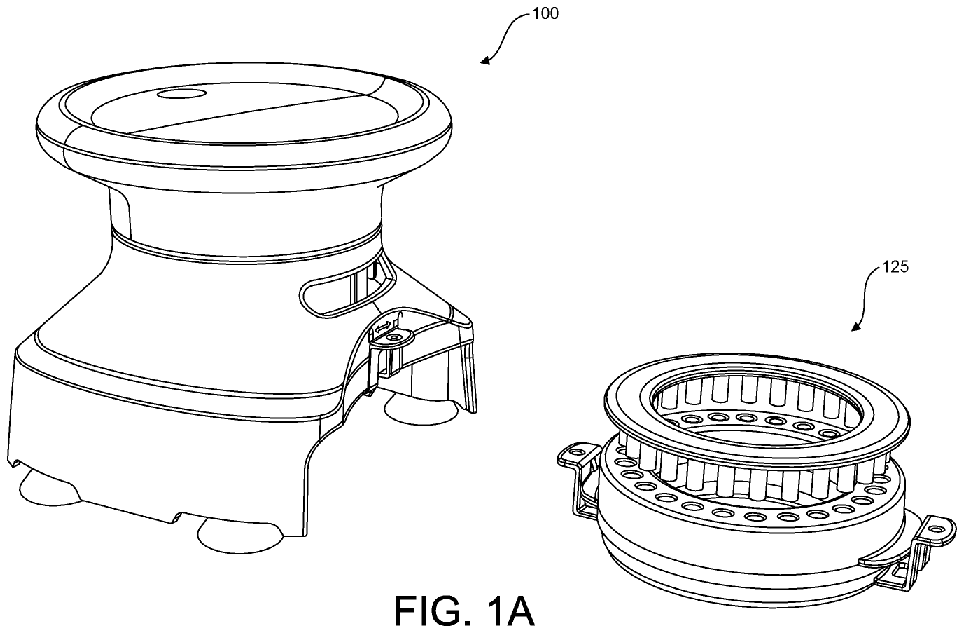

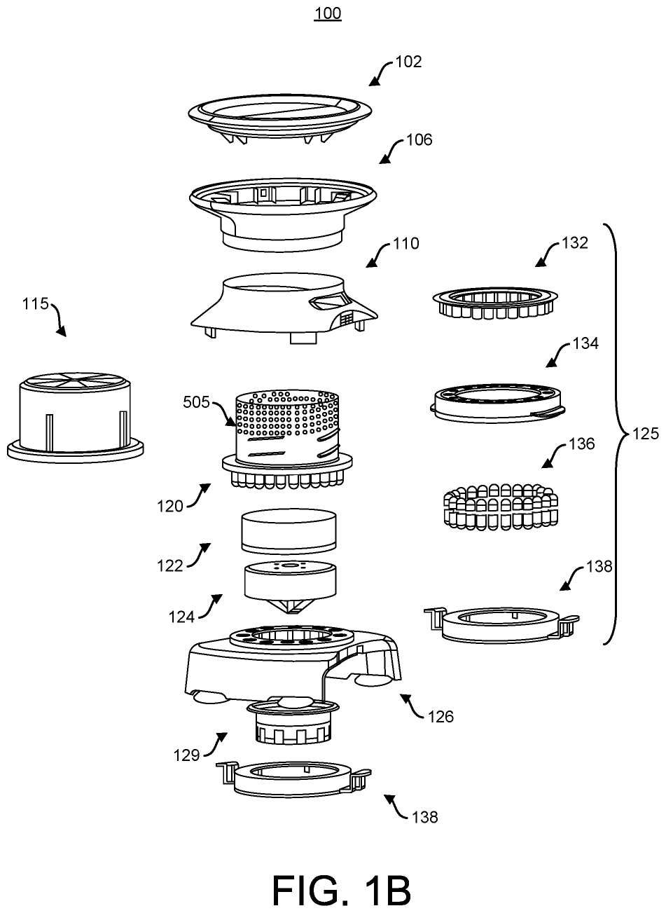

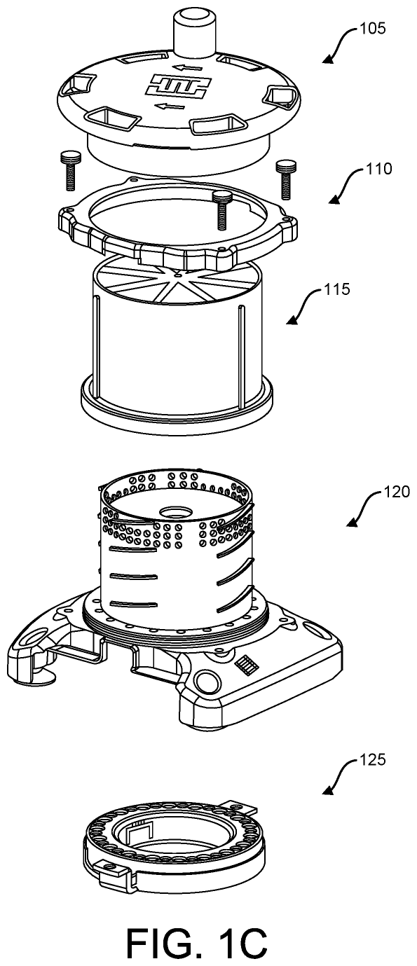

FIG. 1A shows a perspective view of a first embodiment of a point-of-use sample collection and processing device and system that is configured to collect, store, mix, filtrate, and/or encapsulate a stool sample (which can also be referred to as a fecal sample.) The system includes a device 100 that can store, mix, filtrate and/or encapsulate a stool sample wherein the device 100 is a self-contained structure that can perform one or all these features. As described below, the device includes a container that can capture and store a stool sample and a processing component such as a piston and filter that can process, mix, and filter the stool sample as well as transfer the stool sample or portion thereof into one or more capsules. The processing component can include a structure of the device (which can be a force exertion element such as a piston) that exerts a force onto the sample to push or otherwise move at least a portion of the sample toward and into the capsules along a pathway that can include a filter and/or an injector nozzle configured to inject the portion of the sample into the capsules or portion thereof. As described below, the device can also dispense portions of the stool sample into one or more capsule(s). The mixing and dispensing mechanism can include, for example, an auger mechanism, a piston, and an injection nozzle as well as a crank that is configured to be actuated to process the sample. It should be appreciated that the piston may be an optional feature and that the device can incorporate other structures for exerting a force onto the sample to transfer at least a portion of the sample through the device and into capsule or portion thereof.

With reference still to FIG. 1A, the system includes a capsule carrier assembly 125 that is configured to store, contain, support, and/or form the capsules or portions thereof. The capsule carrier assembly 125 (or a portion thereof) can be coupled to a main body of the device 100 for forming at least one capsule and injecting a portion of a stool sample (or sample of a material other than a stool) into the at least one capsule (or portion thereof). In an embodiment, one or more components of the system that touch the stool sample are disposable. For example, any portion of the system that contacts the stool sample can be disposable.

FIG. 1B shows an exploded view of the system of FIG. 1A. The components can be mechanically assembled to one another to collectively form the sample collection device 100. The device 100 includes a crank cover 102 that forms a top region of the device 100. The crank cover 102 couples to and covers a crank body 106 that can be cranked or otherwise actuated to process a stool sample (such as by mixing and/or filtering the stool sample), as described below. An annular retaining ring 110 couples to the crank body 106 and forms at least a portion of an outer shell body of the device 100. A container 115 couples to the base 110 and is held in place by the retaining ring 110. The container 115 is configured to receive and store a stool sample wherein at least one or more portions of the sample can be mixed and transferred to one or more ingestible capsules, as described below.

With reference still to FIG. 1B, a cartridge 120 couples to the retaining ring 110. The cartridge forms a cylindrical (or other shaped) piston chamber that contains a movable piston 122 and a piston actuator 124 configured to mechanically interact with the piston 122 and the crank body 106 to cause movement of the piston 122 within the piston chamber of cartridge 120 for processing the stool sample positioned with the container 115. A base member 126 forms a base of the device 100 to enable the device 100 to be positioned atop a surface, such as a table. The base member 126 can include one or more feet that support the device atop a surface. A piston dial 129 is positioned on or in the base member 126 and is configured to at least partially control actuation of the piston 122.

A capsule carrier assembly 125 is configured to hold or retain one or more portions of a capsule for subsequent receipt of the stool sample once the stool sample has been processed by the device. The processing of the stool sample can include mixing and filtering of the sample for example. In the embodiment of FIG. 1B, the capsule carrier assembly 125 includes several components that mechanically attach to one another to form the capsule carrier assembly 125. In this regard, the capsule carrier assembly 125 includes a capsule ejector 132, a capsule capper 134, a collection of one or more capsules 136 (or portions thereof), and a capsule carrier 138. The capsule carrier 138 is shown twice in FIG. 1B to indicate that the capsule carrier 138 can alone be attached to the device during processing of the stool sample. The capsule carrier 138 can then be attached to the other components of the capsule carrier assembly 125 for formation of capsules, as described below.

The one or more capsules can initially be provided in a multi-part part format such as a first capsule portion that mates with a second capsule portion to collectively form a capsule. For example, the first capsule portion may be a capsule cap portion of a capsule that mates with a capsule body portion (such as in a male-female relationship) of a capsule to form a single capsule having a cavity that contains a portion of stool sample. The capsule cap portion may be a top portion of the capsule while the capsule body portion is a bottom portion of the capsule. Thus, the capsules can initially be configured as a partial portion (e.g., half portion) of a capsule (such as a half capsule) wherein the half portion (or other portion that is not necessarily a half) are assembled to form a full capsule that contains a sample.

### Alternate Embodiment of Device

FIG. 1C shows an exploded view of a second, or alternate, embodiment of the sample collection device 100 that is configured to collect, store, mix, filtrate, and/or encapsulate a stool sample. As in the embodiment of FIGS. 1A and 1B, the device includes components that are configured to receive, process and package a stool sample such as in the form of a fecal sample contained in at least one capsule that is sized and shaped to be ingested by a user in an oral form and/or via suppository. It should be appreciated that both embodiments are configured to process the stool sample and that components of one embodiment may be interchangeable with another embodiment. Furthermore, the description of one embodiment is applicable to another embodiment unless otherwise noted.

In the example embodiment shown in FIG. 1C, the device includes components comprising a crank 105, a retaining ring 110, a container 115, a cartridge 120, and a capsule carrier assembly 125. The components can be mechanically assembled to one another to collectively form the sample collection device 100. The container 115 is configured to receive and store a stool sample wherein at least one or more portions of the sample can be mixed and transferred to one or more ingestible capsules, as described below. The capsule carrier assembly 125 is configured to hold or retain one or more portions of a capsule for subsequent receipt of the stool sample once the stool sample has been processed by the device.

### Capsule Carrier Assembly

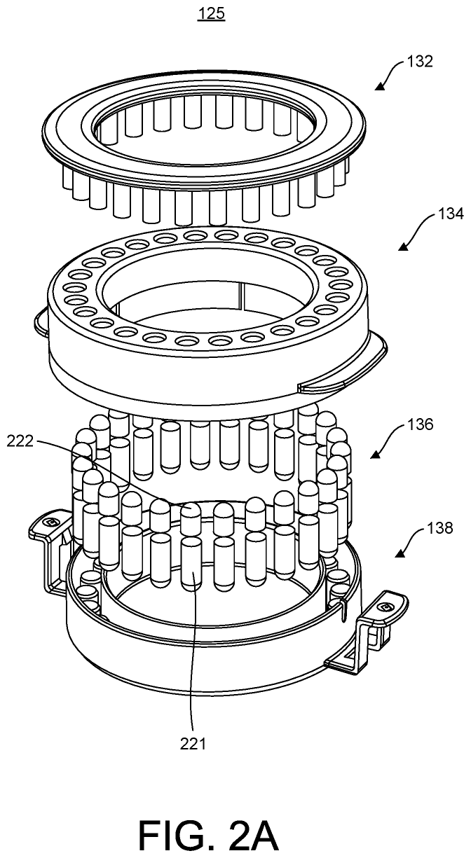

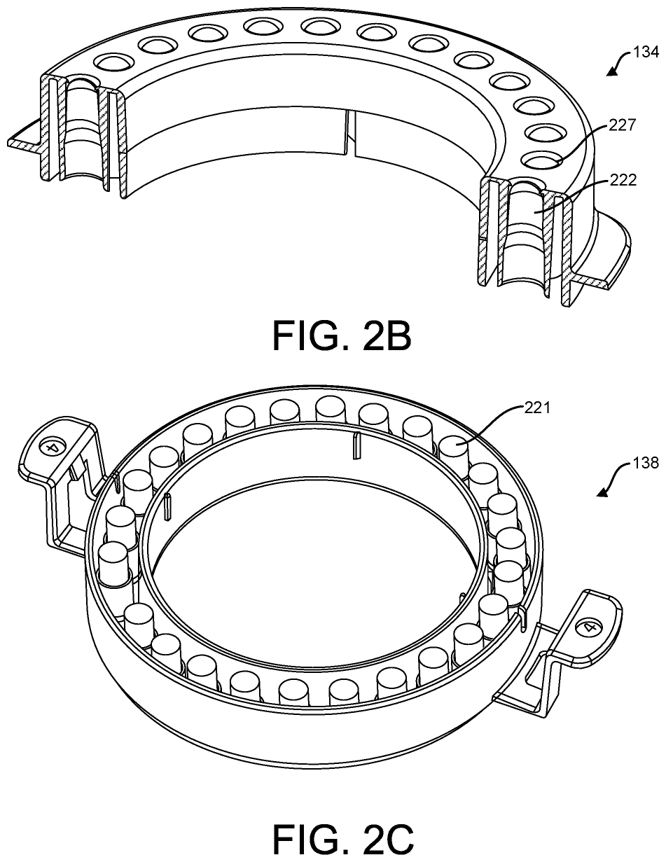

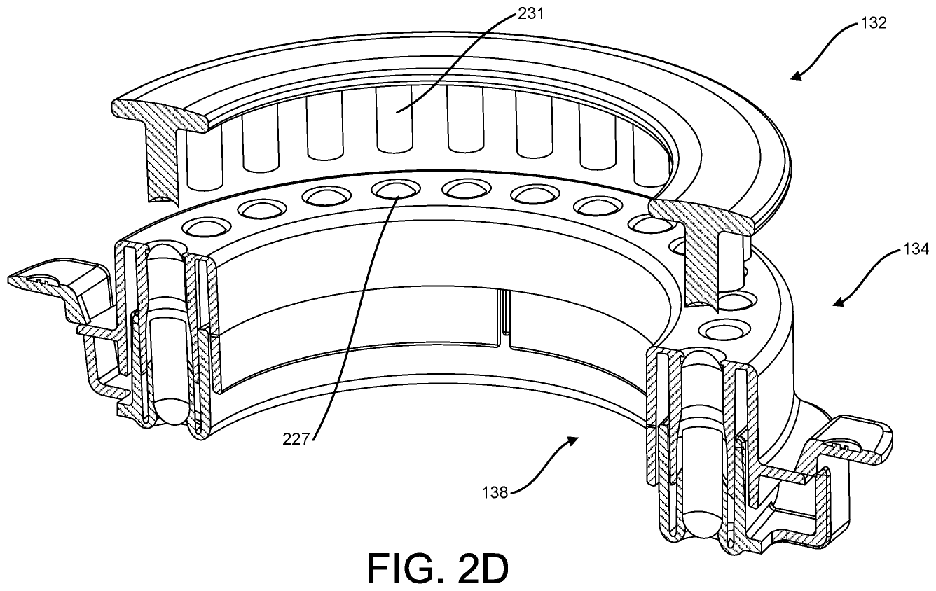

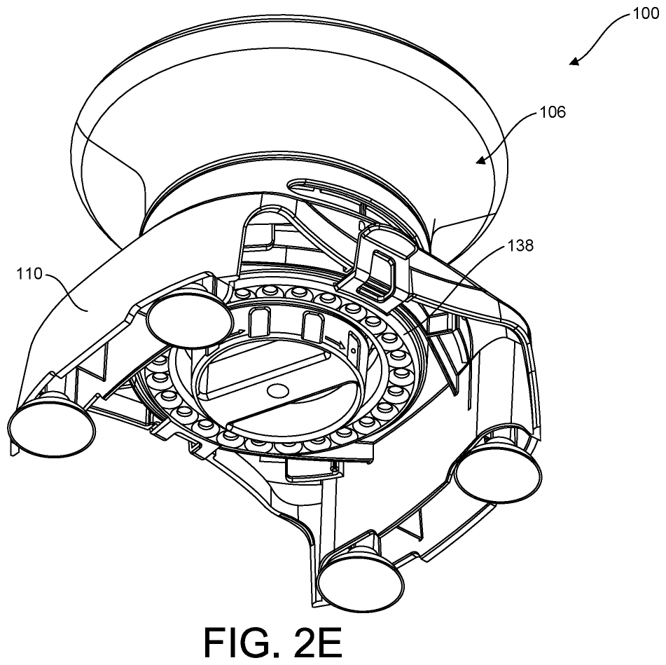

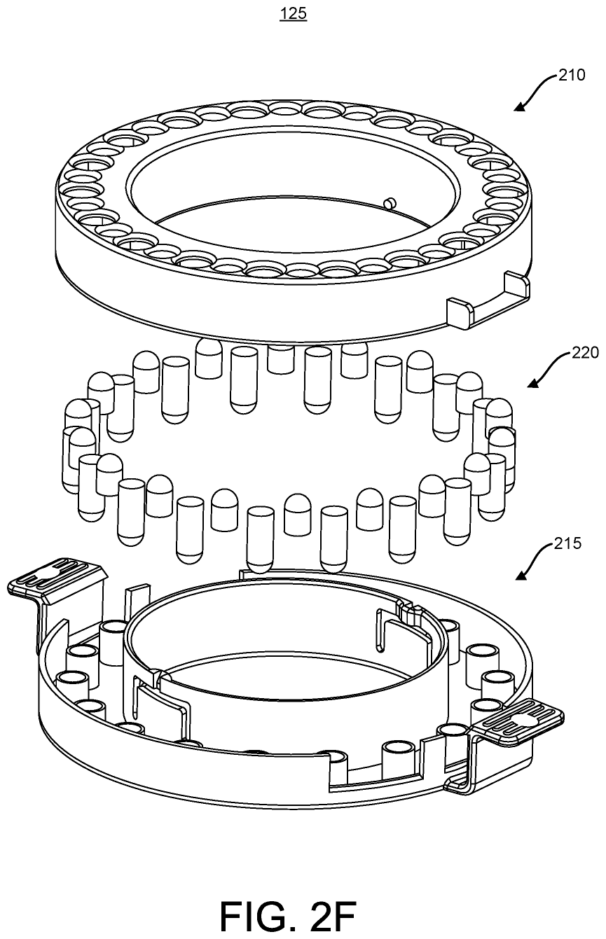

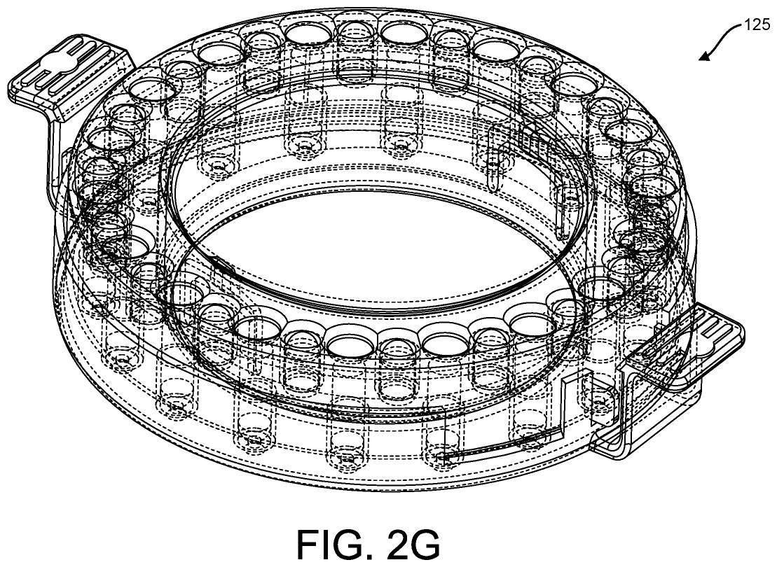

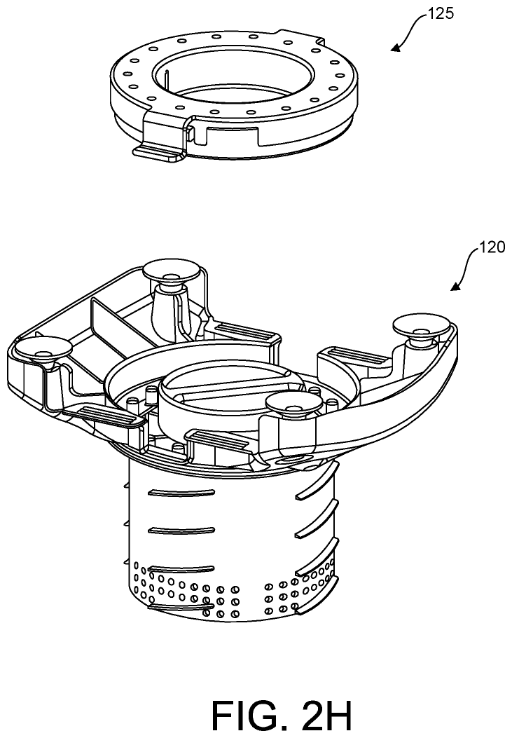

FIG. 2A shows an exploded view of a first embodiment of the capsule carrier assembly 125, which is configured for use with the embodiment of the device shown in FIGS. 1A and 1B. As mentioned, each of the capsules 136 is provided in the form of a capsule body portion 221 that mates with a capsule cap portion 222 to collectively form an entire capsule. The capsule carrier 138 is a ring-like or annular structure that supports a plurality of capsule body portions 221 along a perimeter of the capsule carrier 138. The capsule capper 134 supports or contains a plurality of capsule cap portions 222 in a spatial arrangement that corresponds to or complements the spatial arrangement of the capsule body portions 221. Each capsule body portion 221 has an aligned capsule cap portion 222 when the capsule capper 134 is coupled atop the capsule carrier 138.

FIG. 2B shows the capsule capper 134 in cross-section and shows a capsule cap portion 222 positioned in each of a plurality of seats of the capsule capper 134. An aperture or port 227 is aligned atop each of the seats in alignment with a capsule cap portion 222 positioned in the respective seat. FIG. 2C shows the capsule carrier 138 with a plurality of capsule body portions 221 seated in an annular arrangement (or other spatial arrangement) that complements an annular arrangement of the capsule cap portions of the capsule capper 134. As mentioned, when the capsule capper 134 is coupled to the capsule carrier 138, each capsule cap portion 222 aligns with a respective capsule body portion 221. The capsule capper 134 and the capsule carrier 138 thus assist in proper and simultaneous alignment of a plurality of capsule cap portions 222 with a corresponding plurality of capsule body portions 221 and subsequent formation of a plurality of capsules upon full attachment of the capsule capper 134 to the capsule carrier 138. As shown in FIG. 2D, the capsule ejector 132 is a comb-like structure having a plurality of prongs 231 arranged in an annular arrangement with each prong 231 aligning with a corresponding port 227 as well as a corresponding capsule cap portion and capsule body portion.

As described further below, the capsule carrier 138 can be mechanically attached to the cartridge 120 in a fill configuration, as shown in FIG. 2E. The piston 122 and the piston actuator 124 (FIG. 1B) are also coupled to the cartridge 120 and the container 115 such that the device can process the sample and deliver or fill a portion of the stool sample into the capsule body portions 221 (positioned in the capsule carrier 138) via nozzles that interact with the ports 227 (FIG. 2B). The nozzles are positioned such that each nozzle aligns with a respective capsule body portion 221 when the capsule carrier 138 is coupled to the device. The nozzles then serve as a conduit for transferring the processed stool sample into a plurality of (or at least one) capsule body portions 221. The capsule body portions can then be mated with the capsule cap portions by removing the capsule carrier 138 from the device and then coupling the capsule capper 134 to the capsule carrier 138. The capsule carrier 138 has a plurality of openings (one for each capsule) in a bottom portion of the capsule carrier.

### Alternate Embodiment of Capsule Carrier Assembly

FIG. 2F shows an exploded view of a second embodiment of the capsule carrier assembly 125 that is configured to hold one or more capsules or portions thereof for storing a sample in connection with the alternate embodiment of the device shown in FIG. 1C. It should be appreciated that the figures are examples and that embodiments can vary including the quantity and size of capsules. The alternate embodiment of the capsule carrier assembly 125 is formed of one or more carrier portions including a first carrier portion 210, a second carrier portion 215 and an array of mating capsules 220 including capsule cap portions and capsule body portions. The first carrier portion 210 and second carrier portion 215 mate with one another and contain or support respective capsule body portions and capsule cap portions. Each capsule body portion mates with a capsule cap portion to form an assembled capsule in which a stool sample can be contained. In the illustrated embodiment, the carrier assembly 125 and its portions are annular although the shape may vary.

The capsule body portions are positioned or loaded into the first carrier portion 215. Each capsule body portion fits into a seat of the first carrier portion 215 wherein each seat is sized and shaped to receive a respective capsule body portion. Each capsule cap portion fits into a respective seat in the second carrier portion 210. The seat is sized and shaped to receive a respective capsule cap portion. The capsule cap portions are loaded into the second carrier portion 210 such that each of a plurality of capsule cap portion can align with a corresponding capsule body portion of the first carrier portion 215. In a step of the sample processing sequence, the capsule body portion portions are positionally offset (such as relative to a circumference of the first and second carrier portions) from the capsule cap portions and can be subsequently repositioned into an aligned state such that each capsule portion can be coupled to form a complete capsule. With the base half capsules and top half capsules loaded on their respective carriers, the first carrier portion 215 and the second carrier portion 210 are snapped together to form the capsule carrier assembly 125, which captures at least one capsule body portion and capsule cap portion. In an embodiment, the capsule carrier assembly 125 comes to the user pre-loaded with capsules and in an assembled state. It is oriented in the “fill” configuration, as shown in FIG. 2G, which shows the capsule carrier assembly 125 in an assembled state.

FIG. 2G shows the alternate embodiment of the capsule carrier assembly 125 in a fill configuration wherein the capsule portions (the capsule cap portions and the capsule body portions) are arranged and contained in an alternating array around the perimeter of the capsule carrier assembly 125. The capsule body portions are oriented with an open side up and are aligned with corresponding openings in the first carrier portion 215 (or second carrier portion.) This allows fill nozzles of the device 100 to extend into the capsule body portions via the openings in the first carrier portion 215 for filling, as described more fully below.

### Sample Collection and Processing

In use, the container is used to capture a stool sample from a user, as described more fully below. The container (which contains a stool sample) is then coupled to the device 100, such as by mechanically attaching the container to device 100, which also includes the piston and filter. The piston is actuated to process the stool sample, such as by exerting a force or a series of forces onto the stool sample, which can cause the stool sample to be pushed through one more filter elements within the device 100. As the piston exerts a force on the stool sample (or before or after the piston exerts the force), the sample can also be mixed such as through use of an auger assembly as described below. The piston can also exert a force that pushes portion(s) of the stool sample into the capsule portions positioned in the capsule carrier assembly via one or more injection ports or nozzles that communicate with the capsule carrier assembly when the capsule carrier assembly (or portion thereof) is attached to the device. As mentioned, the device includes a quantity of injection nozzles that corresponds to a quantity of capsules (or capsule portions) with the injection nozzles in a spatial arrangement that aligns each nozzle with a corresponding capsule or capsule portion. The nozzles are configured to simultaneously and automatically inject a portion of the stool sample into each of or one or more of the capsule or capsule portions. A user can then eject the capsules from the capsule carrier assembly for storage or immediate use.

In preparation for sample collection using the embodiment of the device shown in FIGS. 1A and 1B, the capsule carrier assembly 125 is coupled to the cartridge 120 in a fill configuration, as shown in FIG. 2E. The cartridge 120 is sized and shaped to receive and mate with the capsule carrier 138 such as by the capsule carrier 138 mechanically fitting onto or into a portion of the cartridge 120. That is, the capsule carrier 138 is positioned such that each of the capsule body portions aligns with a respective nozzle when the capsule carrier 138 is mated with the device 100. The capsule carrier 138 and cartridge 120 may form an assembled structure. As mentioned, the cartridge 120 includes one or more fill or injection nozzles, which align with and fluidly communicate with the capsule body portions 221 in the capsule carrier 138. The fill nozzles are configured to transfer at least a portion of the stool sample into respective capsule body portions 221.

Regarding the alternate embodiment (shown in FIG. 1C), FIG. 2H shows the second embodiment of the capsule carrier assembly 125 in a position ready to be coupled to the cartridge 120. The cartridge 120 is sized and shaped to receive and mate with the capsule carrier assembly 125 such as by the capsule carrier assembly 125 mechanically fitting onto a portion of the cartridge 120. The capsule carrier assembly 125 and cartridge will then form an assembled structure. As in the first embodiment, the cartridge 120 includes one or more fill nozzles, which align with the previously mentioned openings of the first carrier portion 210. The fill nozzles protrude through holes in the first carrier portion 210 and into the capsule body portions positioned in the second carrier portion 215. The fill nozzles interface with the capsule body portion portions and are configured to transfer at least a portion of the stool sample into respective capsule body portions. Once the capsule carrier assembly 125 is snapped onto the cartridge 120, the user is ready for stool sample collection using the device 100.

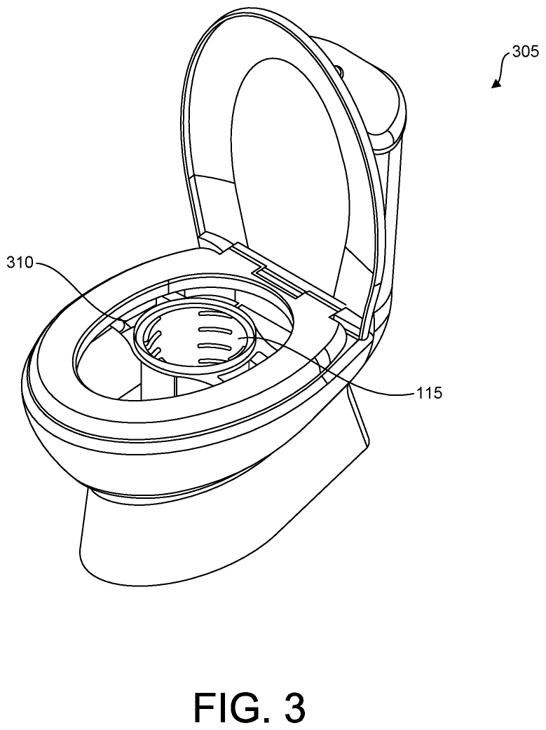

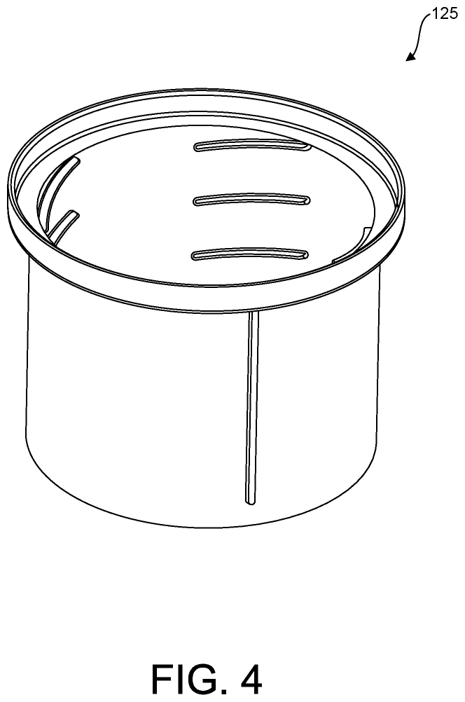

Stool is collected in the container 115 as schematically illustrated in an example shown in FIG. 3. During collection, the container 115, which includes an internal cavity with an open end, is supported in a toilet 305 using a structure 310 to support the container 115 in the center of the toilet 305. A user can sit on the toilet 305 and deposit a stool sample into the container 115. It should be appreciated that the user can use other techniques for depositing the stool sample into the container 115.

FIG. 4 shows the container 115, which is cylindrical as shown although the shape may vary. As mentioned, the container 115 has an internal cavity formed by one or more internal side walls. An auger structure is located around the perimeter of the internal side walls of the container 115. In this embodiment, there are four rows of four augers. However, it should be understood that many variations in size, shape and quantity of the auger structure are within the scope of this disclosure. In addition, the size and shape of the container 115 can vary. A top edge of the container can be stepped to provide a support shoulder on the exterior of the container 115 and a sealing surface on the interior. In a non-limiting example embodiment, the container is approximately 6″ in diameter and 4.5″ tall.

It is noted that the user may evaluate the sample using the Bristol stool index. For proper function, the sample should be in the middle of the Bristol stool index. Samples which are too firm or too soft will be difficult to process and should be discarded.

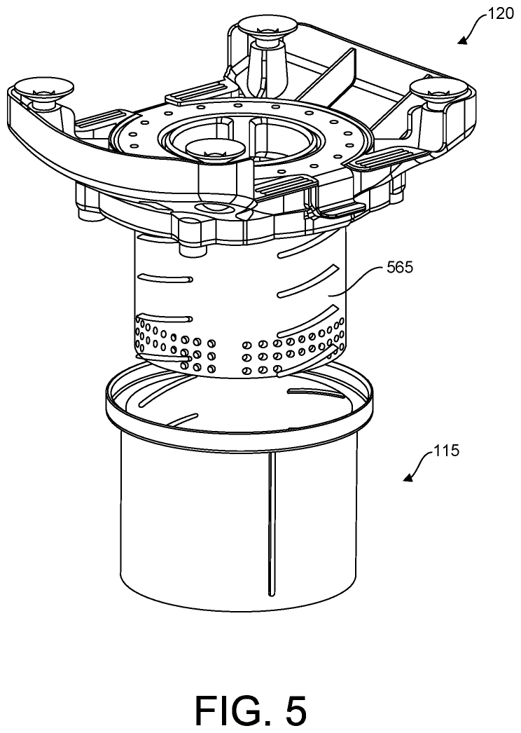

Upon collection of the sample in the container 115, the cartridge 120 is installed into and mated with the container 115 as shown in FIG. 5. FIG. 5 shows the alternate embodiment of FIG. 1C although the container 115 and cartridge 120 mate in a similar manner for the embodiment of FIGS. 1A and 1B. The cartridge 120 can have a cylindrical insertion member 505 that fits into the complementary-shaped cavity of the container 115 such as in a snug manner. The insertion member 505 can have an outer wall with a complementary augur structure that mechanically interacts with the auger structure of the container 115. The device can be actuated to cause relative movement between the insertion member 505 and the container 115 to mechanically process the sample via the auger structures. The device is also configured to cause relative movement (such as rotational movement) between the insertion member 505 and the container 115 so that the augers mix a stool sample positioned therebetween. In an embodiment, the device does not have a piston and the insertion member 505 serves as the force exertion element such that at least a portion of the insertion member 505 exerts a force onto the sample.

In an example embodiment, the container 115 is held in place relative to the cartridge 120 with the retaining ring 110 (FIGS. 1B and 1C) which is held in place with thumb screws or other type of securing member. Another embodiment allows the container to be snapped into place using integral plastic latches which are molded into the container 115 and/or cartridge 120. The container/cartridge interface is rotatably sealed using one or more of seals, such as O-rings in an example embodiment.

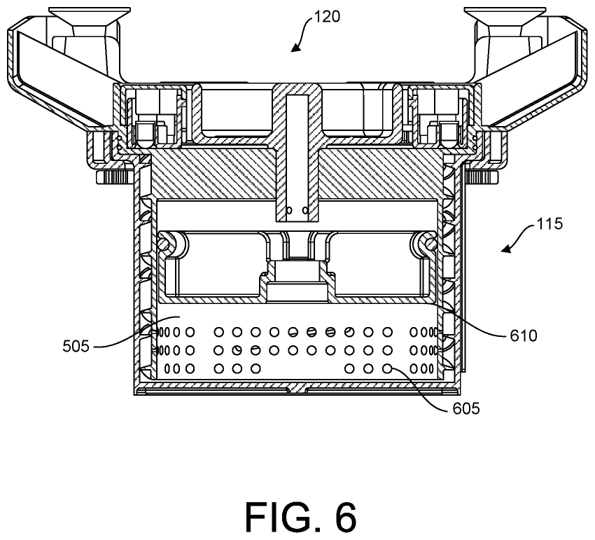

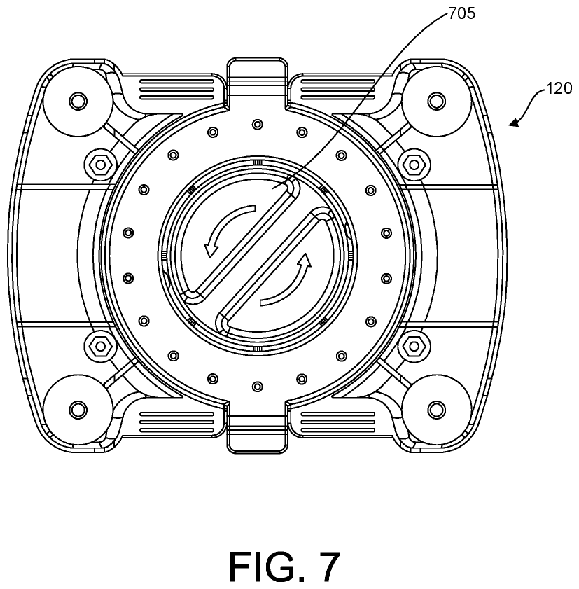

FIG. 6 shows a section view of the assembled container 115 and the cartridge 120 such that the cylindrical insertion member 505 of the container 115 is at least partially positioned within the cartridge 120. FIG. 6 shows this in the context of the alternate embodiment of FIG. 1C. A bottom portion of the assembled container and cartridge includes a filter element such as an array of filter holes 605, which extend through the cylindrical insertion member 505. A movable piston 610 (or piston 122) is positioned above the filter holes 605 within the cylindrical insertion member 505 formed by the cartridge 120 such that the piston can alternately extend into and retract out of the container 115 in a cycling manner. When retracted upward, the piston 610 creates or forms a collection volume for the sample to reside during insertion of the cartridge 120. The collection volume is the volume of space below the bottom end of the piston. A mixing volume is defined by an annular gap between the inside wall of the container 115 and an outside wall of the insertion member 505 of the cartridge 120. The device can include an actuator, such as for example a helical acme threaded feature connected to a dial which actuates the piston 610, such as to extend and/or retract the piston 610 relative to the container. FIG. 7 shows a top view including the actuator (such as the dial 705). As mentioned, components of the device that contact the stool sample can be removable and/or disposable. In an example, the container, cartridge and piston are such components.

Once the cartridge 120 is secured to the container 115 as shown in FIG. 6, the user manipulates an actuator, such as by rotating the piston actuator dial 705 (FIG. 7). As the dial 705 is rotated, a mechanism such as an acme nut connected to the piston is driven downward, thus driving the piston 610 downward toward the filter holes 605 (FIG. 6). It should be appreciated that other mechanisms can be used to actuate, move or drive the piston 610.

As the piston 610 is driven through the device and toward the filter holes, the sample is driven, extruded, or otherwise forced from the collection volume into the mixing volume through the filter holes 605. The filter holes 605 can vary in size. In an embodiment, the filter holes are sized to prevent objects larger than the fill nozzles to enter the mixing volume. The filter size could be smaller depending on the level of filtering desired. As mentioned, the mixing volume is defined by a gap between the inside wall of the container 115 and an outside wall of the insertion member 505 of the cartridge 120.

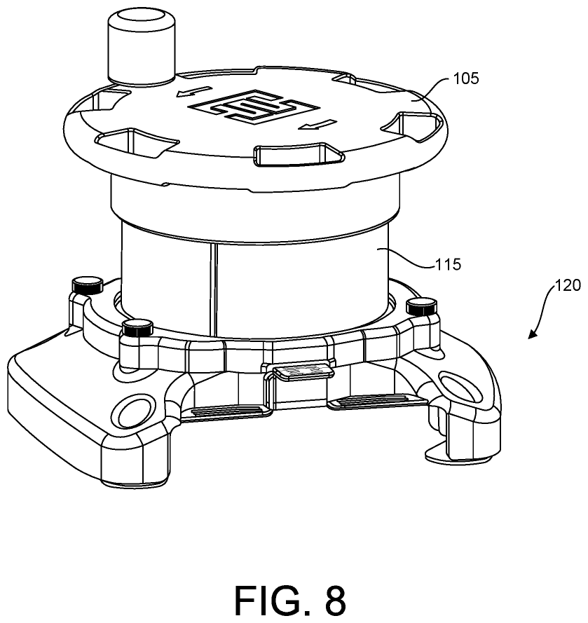

In an embodiment, the container includes an insert molded, translucent viewing window which allows the user to monitor the level of the sample as it extrudes into the mixing volume. Once the sample reaches an indicator (such as a “full” line) on the container 115, the user flips the device over and mounts it to a flat surface using a securing mechanism, such as suction cups or rubber feet positioned on the cartridge 120. At this point, the user installs the crank 105 by sliding it on to the container as shown in FIG. 8.

To continue processing of the collected sample, the user actuates the device by actuating an actuator, such as for example by rotating the crank 105, such as in a counterclockwise direction. The crank 105 is mechanically coupled to the container 115 so that this in turn rotates the container relative to the cylindrical insertion member 505 of the cartridge 120. As mentioned, the cylindrical insertion member 505 of the cartridge 120 has outwardly protruding auger features which interpose or mechanically interact with the auger features on the container wall. The augers on the cylindrical insertion member 505 of the cartridge are arranged at an opposing helix angle to those on the container 115. A relatively narrow gap between the container 115 and the cartridge 120 creates a shear force on the stool sample which causes relative motion between the sample, the container 115 and the cartridge when the container 115 and cartridge 120 are rotated relative to one another or relative rotational movement is achieved.

When the container 120 is rotated in a counterclockwise direction (when device is oriented as shown in FIG. 8), the auger features on the cartridge 120 and the container 115 drive the sample upward. The sample is unable to move back into the filter given the still full collection volume retained by the piston. Thus, the sample recirculates downward, creating a mixing action for the sample. The user can turn the crank a quantity of revolutions (such as ten revolutions) in the CCW direction for mixing in a non-limiting example. It should be appreciated that the quantity of revolutions can vary and can be a function of achieving a desired level of mix for example.

Upon completion of the mixing cycle, the user then optionally reverses the direction of rotation (by actuating the crank 105 in the opposite direction for example). For example, the user can turn the crank 105 a quantity of revolutions (such as 10 times in a non-limiting example) in the clockwise direction. This causes the augers to drive the sample downward and toward one or more fill nozzles positioned at the bottom of the cartridge 120. The device is thus configured to use the auger arrangement to mix the sample such as via rotation or other movement of a first set of auger structured on the container relative to a second set of auger structures on the device, such as on the cartridge.

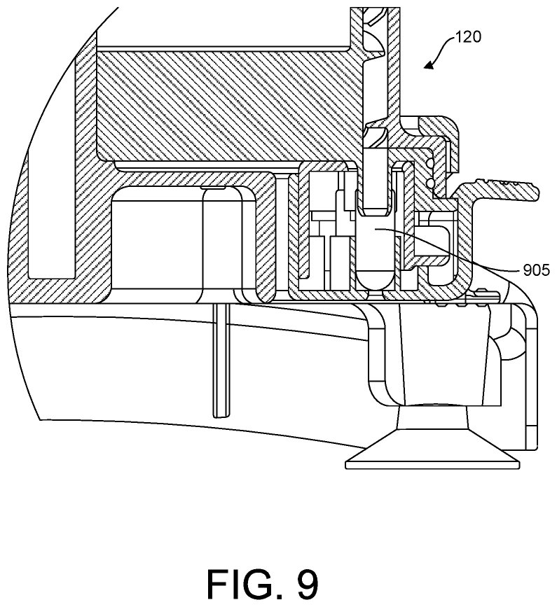

As shown in FIG. 9, fill or injection nozzles 905 of the cartridge 120 protrude into the capsule carrier assembly 125 (or into the capsule carrier 138 in the first embodiment) when it is assembled onto the cartridge 120. The injection nozzles provide a passageway for at least a portion of the sample to be transferred into capsule portion such as a capsule body portion. The nozzles 905 extend about 3 mm into each of the capsule portions in a non-limiting example embodiment. The nozzles 905 are sized to allow air to escape as the capsules are filled. However, in an embodiment, a gap between the capsule and the nozzle is not large enough for the sample to escape. Thus, once the capsule has filled, it creates enough back pressure to resist the pressure created by the augers, so the capsules do not overspill.

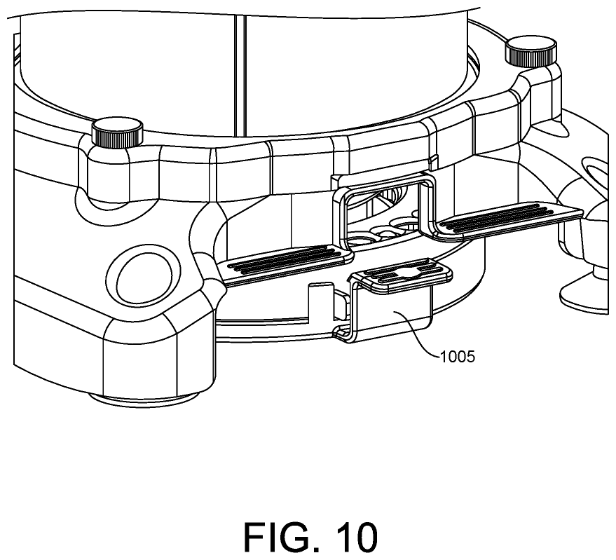

Upon completion of this mixing cycle, the user disconnects the capsule carrier assembly 125 from the cartridge 120 such as by de-coupling or otherwise moving tabs 1005 shown in FIG. 10. For the embodiment of FIGS. 1A and 1B, only the capsule carrier 138 is coupled to the device. The user can then assemble the entire capsule carrier assembly as described below. It should be understood that ability to attach and detach the capsule carrier could be implemented in various embodiments by tabs, threads, friction fits, or bayonet style twist lock mating features similar to electrical connects, jar lids, and beverage covers.

For the embodiment of the capsule carrier assembly 125 shown in FIGS. 1A, 1B, and 2A-2E, the capsule carrier 138 is now detached from the device 100. At this point, the capsule body portions 221 each contain a portion of the stool sample. The user then positions the capsule capper 134 (which contains the capsule cap portions 222) atop the capsule carrier 138 and snaps the capsule capper 134 onto the capsule carrier 138. This causes each of the capsule cap portions 222 to mate with a respective capsule body portion 221 to thereby form an assembled capsule, which contains a portion of the stool sample. As shown in the cross-sectional view of FIG. 2D, the user can then use the capsule ejector 132 to eject the assembled capsules by aligning each prong 231 with a respective port 227 and pushing the prongs 231 into the port so that the prongs eject the capsules from the capsule carrier 138 via a respective opening in the bottom of the capsule carrier 138.

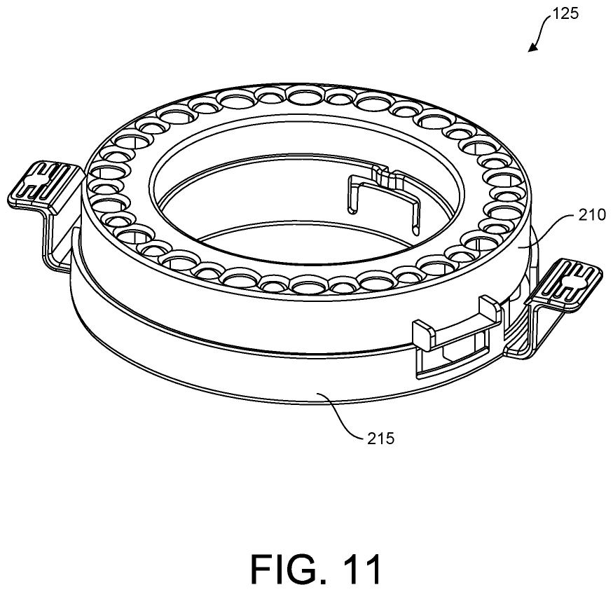

Regarding the capsule carrier assembly 125 used with the second embodiment of the device, the capsule carrier assembly 125 with the capsules can be slid out or otherwise detached from the device 100. If desired, a second capsule carrier assembly 125 can then be installed. The user then actuates the device (such as by turning another 10 clockwise cranks in a non-limiting example) thus filling the second capsule carrier assembly 125. Additional capsule carrier assemblies may be inserted and filled until the mixing volume is depleted to the point it will no longer dispense. When the capsule carrier assembly 125 is removed from the device, the user lifts upward on the second carrier portion 215 and rotates the second carrier portion 215 relative to the first carrier portion 210. FIG. 11 shows a perspective view of the capsule carrier assembly 125. A track on the inner wall of the first carrier portion 210 can be configured to guide the user through this motion.

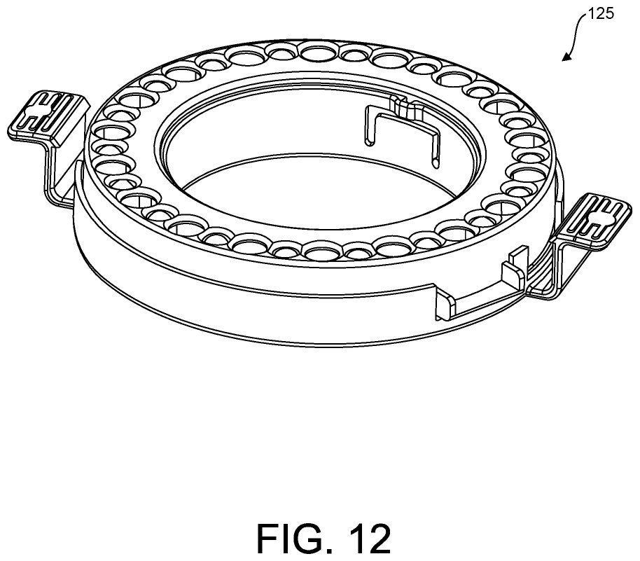

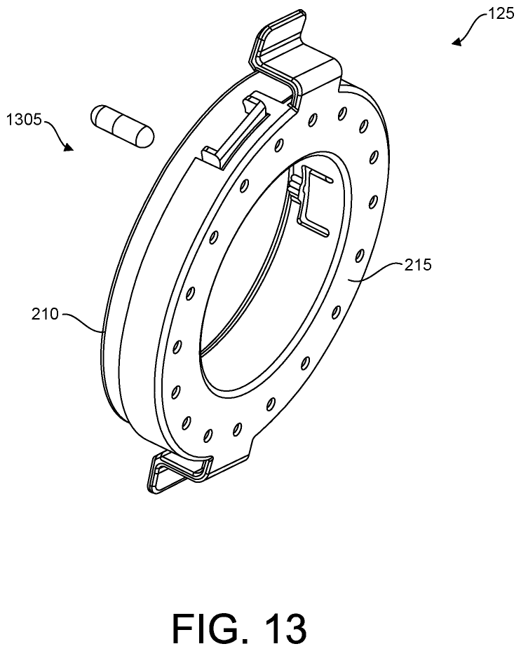

Once rotated, the capsule cap portions and capsule body portions within their respective carrier portions are now vertically aligned wherein the capsule cap portions and capsule body portions were previously offset from one another along the circumference of the capsule carrier assembly 125. The capsule cap portions and capsule body portions are thus in positions to be coupled to one another to form a plurality of complete capsules. The user then simply pushes the first carrier portion 210 downward toward the second carrier portion 215 to thereby mate the capsule cap portions with the respective capsule body portions, closing and sealing the capsules. At this point, the capsule carrier assembly 125 (shown in FIG. 12) can transferred to a freezer such as at an example temperature of −20 C or −80 C. Once the sample is frozen within the capsules in the capsule carrier assembly 125, the capsules 1305 may be removed from the cartridge such as by pushing a removal tool 1310 through one of the small holes on the back of the first carrier portion 210 to push the capsules outward as shown in FIG. 13.

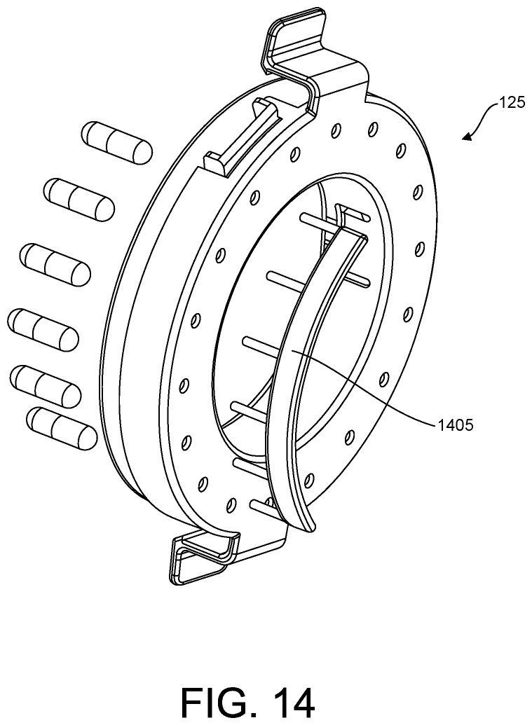

In the event that a user wishes to eject multiple capsules, an ejection comb 1405 can be used as shown in FIG. 14. The comb 1405 includes a plurality of protrusions that respectively align with the seats in the capsule carrier assembly 125 in which the capsules are positioned. The protrusions can be simultaneously inserted into the multiple seats to eject multiple capsules from the capsule carrier assembly 125.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a sub-combination or a variation of a subcombination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. An apparatus for collection and processing of a stool sample comprising:
a container defining a cavity that can contain a stool sample;
a capsule carrier assembly defining at least one seat that supports a capsule portion;
at least one fill nozzle mechanically coupled to the capsule carrier assembly and the container, wherein the fill nozzle provides a passageway for a portion of the stool sample to be transferred from the container into the capsule portion;
a force exertion element mechanically coupled to the container such that the force exertion element exerts a force onto the stool sample in the container to move the portion of the stool sample into the capsule portion via the at least one nozzle; and
a filter positioned relative to the force exertion element, wherein the portion of the stool sample passes through the filter prior to being moved into the capsule portion.

2. The apparatus of claim 1, wherein the capsule carrier assembly supports a plurality of capsule portions positioned in a spatial arrangement.

3. The apparatus of claim 2, wherein the at least one fill nozzle includes a plurality of fill nozzles arranged in a spatial arrangement that complements the spatial arrangement of the plurality of capsule portions.

4. The apparatus of claim 3, wherein the capsule portions are positioned in an annular spatial arrangement.

5. The apparatus of claim 1, wherein the capsule carrier assembly comprises:
a capsule carrier that supports a plurality of capsule body portions; and
a capsule capper that supports a plurality of capsule cap portions such that each capsule cap portion aligns with a corresponding capsule body portion when the capsule capper is coupled to the capsule carrier.

6. The apparatus of claim 5, wherein the capsule carrier assembly further comprises a capsule ejector having a plurality of prongs that interact with the capsule carrier or the capsule capper to eject the capsule body portions and capsule cap portions.

7. The apparatus of claim 5, wherein only the capsule carrier couples to the container.

8. The apparatus of claim 1, wherein the capsule carrier assembly is annular.

9. The apparatus of claim 1, further comprising a cartridge having a cylindrical insertion member that fits into the cavity of the container.

10. The apparatus of claim 1, wherein the force exertion element is a piston.

11. The apparatus of claim 9, wherein the force exertion element is a piston positioned inside the cylindrical insertion member of the cartridge.

12. The apparatus of claim 9, wherein the force exertion element is the cylindrical insertion member.

13. The apparatus of claim 1, wherein the container rotates relative to at least an auger portion of the device to mix the stool sample.

14. An apparatus for collection and processing of a stool sample comprising:
a container defining a space that can contain a stool sample;
at least one capsule carrier assembly configured to support at least one capsule;
a cartridge including a piston, a filter, a mixing and dispensing mechanism, and one or more fill nozzles configured to align with an opening of the at least one capsule, wherein the cartridge couples to the at one container; and
an actuator that can be actuated to rotate the cartridge.

15. The apparatus of claim 14, wherein at least a portion of the container can achieve rotational movement relative to the cartridge to mix the stool sample.

16. The apparatus of claim 14, where the piston is configured to displace the stool sample through the filter.

17. The apparatus of claim 14, where the mixing and dispensing mechanism comprises at least one auger.

18. The apparatus of claim 17, wherein the at least one auger is a first auger on an exterior surface of the cartridge.

19. The apparatus of claim 17, wherein the at least one augur is a second auger on an interior surface of the container.

20. The apparatus of claim 14, wherein the capsule carrier comprises:
a first capsule portion and a second capsule portion arranged in offset positions relative to one another;
a mechanism configured to align the first capsule portion with the second capsule portion; and
a guide to axially displace the portions to mate the first capsule portion and a second capsule portion to form a closed capsule.

21. The apparatus of claim 14, wherein the actuator is a hand crank configured to cause sample mixing in one direction and sample dispensing in a second direction.

22. The apparatus of claim 14, wherein the at least capsule carrier assembly is annular.

23. A method of processing a stool sample, comprising:
exerting a force onto a stool sample such that the stool sample passes through a filter of a collection device;
causing the stool sample to pass through a plurality of injection nozzles into a plurality of first capsule portions positioned in or on the collection device, such that each first capsule portion of the plurality of first capsule portions contains a portion of the stool sample;
attaching a second capsule portion to each of the first capsule portions to form plurality of capsules that each contains a portion of the stool sample.

24. The method of claim 23, further comprising removing the plurality of first capsule portions from the collection device prior to attaching the second capsule portions to the first capsule portions.

25. The method of claim 23, further comprising mixing the stool sample using an auger while the stool sample is in the collection device.

26. The method of claim 23, wherein the injection nozzles are positioned in an annular arrangement that corresponds to an annular arrangement of the first capsules.

27. An apparatus for collection and processing of a sample comprising:
a container defining a cavity that can contain a sample;
a capsule carrier assembly defining at least one seat that supports a capsule portion;
at least one fill nozzle mechanically coupled to the capsule carrier assembly and the container, wherein the fill nozzle provides a passageway for a portion of the sample to be transferred from the container into the capsule portion;
a force exertion element mechanically coupled to the container such that the force exertion element exerts a force onto the sample in the container to move the portion of the sample into the capsule portion via the at least one nozzle; and
a filter positioned relative to the force exertion element, wherein the portion of the sample passes through the filter prior to being moved into the capsule portion.

28. The apparatus of claim 27, wherein the sample is a stool sample.
